SEQUENCE LISTING (82 sequences)

SEQ ID NO: 1 - DNA, 1146 bp, Artificial Sequence
  DNA polymerase alpha/primase large subunit with codon usage optimized for expression in E. Coli
atgaaacgtgtaaaaccgctaagaaggtggtggccgggtacaccttctacccgctctgtcaaatcttctgtgactacgaaccttccgtaatgcgctgaactcgtatcgaatgctgcgttggaagaaggtgaggtatcatctcaaatgcctgtcacctctatcgagaagacatccttctcacttctttgccgtgttgttgctctcaggagcttggaacgcgtttggttcgtaacgctgaagtaacctgctgcgctgcgtatctcaaaaacccgggggtgctcgtgcttcttcctgtcgaagttctgcccacatggacgggctcgtacccagacggtgttctgttctgggtatcgttctcgttaaacccggacgtgttgcttctgcgcttcttctgagttctggttcattcaccaaagtatcgacctgatggtaaacgtgcgttgttccgaaggtggttactgcgtctgacgacgaaggttcggttctctctggtttctgattccgtcgtacctgtctaacgtatggacgactggttgaatctcttctaagaaccggacgacgtatgcgtttctgtctacctctctgctggctaacccgaccggttacgaacgtggtaccaacttctctctggaatcttctgaacgttacttcccgccgtgcatgcaggacatcatggaacgtctgcgtaaaaacaaacacctgaaatacaacgaccgtcagaccctgtgcctgttcctgaaagactgcggtatgtctgttgaagacggtatcgctttcttccgtggttctttcaaagctccgcgtgaagttttcgacaaagaatacctgtactctatccgtcacaactacggtctggaaggtaaacgtgctaactactcttgcttctcttgctctaaaatcgctaacatgaccaacgaagaacgtcgttcttcttgcccgttcgttggtgacccggaacacatccgtgaacgtatcaaagacgttggtgctgacatcgaagacatcatgggtgaaggtccgttcaacggtaaatgcacccgtctgctggaaaaactgacctgccgtaaacagtctcgtctggttgctaccccggttcgttactacctggaatgcaaagacccggaacgtaacggtggtgaaatctaa

SEQ ID NO: 2 - protein, 399 aa, Encephalitozoon cuniculi
MKRAKTAKEGGWPGITFYTALSKSSVDYETFRKCAERRIEMLRCEEGDGIISKCLSTSIEEDILSHFFCRVVCSQDAWNAVWFVNAEVNLLRLRISKNPGGARAFFLSEVLPHMDGARTQDGVLVLGMRSRYNPDVVASAASSEVLVHFTKVIDLIGKRAVVPEGGYLRLNDEGIGSLLVSEFRRYLSNRMDELVEISSNEPDERMRVLSTSLLANPTGYERGTNFSLESSERYFPPCMQDIMERLRKNKHLKYNDRQTLCLFLKDCGMSVEDGIAFFRGSFKAPREVFDKEYLYSIRHNYGLEGKRANYSCFSCSKIANMTNEERRSSCPFVGDPEHIRERIKDVGADIEDIMGEGPFNGKCTRLLEKLTCRKQSRLVATPVRYYLECKDPERNGGEI

SEQ ID NO: 3 - DNA, 36 bp, Artificial Sequence
  constructing oligonucleotide
atgaaacgtgctaaaaccgctaaagaaggtggttgg

SEQ ID NO: 4 - DNA, 25 bp, Artificial Sequence
  constructing oligonucleotide
ccgggtatcaccttctacaccgctc

SEQ ID NO: 5 - DNA, 35 bp, Artificial Sequence
  constructing oligonucleotide
tgtctaaatcttctgttgactacgaaaccttccgt

SEQ ID NO: 6 - DNA, 33 bp, Artificial Sequence
  constructing oligonucleotide
aaatgcgctgaacgtcgtatcgaaatgctgcgt

SEQ ID NO: 7 - DNA, 29 bp, Artificial Sequence
  constructing oligonucleotide
tgcgaagaaggtgacggtatcatctctaa

SEQ ID NO: 8 - DNA, 32 bp, Artificial Sequence
  constructing oligonucleotide
atgcctgtctacctctatcgaagaagacatcc

SEQ ID NO: 9 - DNA, 29 bp, Artificial Sequence
  constructing oligonucleotide
tgtctcacttcttctgccgtgttgtttgc

SEQ ID NO: 10 - DNA, 25 bp, Artificial Sequence
  constructing oligonucleotide
tctcaggacgcttggaacgctgttt

SEQ ID NO: 11 - DNA, 34 bp, Artificial Sequence
  constructing oligonucleotide
ggttcgttaacgctgaagttaacctgctgcgtct

SEQ ID NO: 12 - DNA, 29 bp, Artificial Sequence
  constructing oligonucleotide
gcgtatctctaaaaacccgggtggtgctc

SEQ ID NO: 13 - DNA, 28 bp, Artificial Sequence
  constructing oligonucleotide
gtgctttcttcctgtctgaagttctgcc

SEQ ID NO: 14 - DNA, 25 bp, Artificial Sequence
  constructing oligonucleotide
gcacatggacggtgctcgtacccag

SEQ ID NO: 15 - DNA, 39 bp, Artificial Sequence
  constructing oligonucleotide
gacggtgttctggttctgggtatgcgttctcgttacaac

SEQ ID NO: 16 - DNA, 24 bp, Artificial Sequence
  constructing oligonucleotide
ccggacgttgttgcttctgctgct

SEQ ID NO: 17 - DNA, 33 bp, Artificial Sequence
  constructing oligonucleotide
tcttctgaagttctggttcacttcaccaaagtt

SEQ ID NO: 18 - DNA, 31 bp, Artificial Sequence
  constructing oligonucleotide
atcgacctgatcggtaaacgtgctgttgttc

SEQ ID NO: 19 - DNA, 25 bp, Artificial Sequence
  constructing oligonucleotide
cggaaggtggttacctgcgtctgaa

SEQ ID NO: 20 - DNA, 37 bp, Artificial Sequence
  constructing oligonucleotide
cgacgaaggtatcggttctctgctggtttctgaattc

SEQ ID NO: 21 - DNA, 32 bp, Artificial Sequence
  constructing oligonucleotide
cgtcgttacctgtctaaccgtatggacgaact

SEQ ID NO: 22 - DNA, 29 bp, Artificial Sequence
  constructing oligonucleotide
ggttgaaatctcttctaacgaaccggacg

SEQ ID NO: 23 - DNA, 30 bp, Artificial Sequence
  constructing oligonucleotide
aacgtatgcgtgttctgtctacctctctgc

SEQ ID NO: 24 - DNA, 28 bp, Artificial Sequence
  constructing oligonucleotide
tggctaacccgaccggttacgaacgtgg

SEQ ID NO: 25 - DNA, 30 bp, Artificial Sequence
  constructing oligonucleotide
taccaacttctctctggaatcttctgaacg

SEQ ID NO: 26 - DNA, 26 bp, Artificial Sequence
  constructing oligonucleotide
ttacttcccgccgtgcatgcaggaca

SEQ ID NO: 27 - DNA, 30 bp, Artificial Sequence
  constructing oligonucleotide
tcatggaacgtctgcgtaaaaacaaacacc

SEQ ID NO: 28 - DNA, 27 bp, Artificial Sequence
  constructing oligonucleotide
tgaaatacaacgaccgtcagaccctgt

SEQ ID NO: 29 - DNA, 38 bp, Artificial Sequence
  constructing oligonucleotide
gcctgttcctgaaagactgcggtatgtctgttgaagac

SEQ ID NO: 30 - DNA, 32 bp, Artificial Sequence
  constructing oligonucleotide
ggtatcgctttcttccgtggttctttcaaagc

SEQ ID NO: 31 - DNA, 29 bp, Artificial Sequence
  constructing oligonucleotide
tccgcgtgaagttttcgacaaagaatacc

SEQ ID NO: 32 - DNA, 30 bp, Artificial Sequence
  constructing oligonucleotide
tgtactctatccgtcacaactacggtctgg

SEQ ID NO: 33 - DNA, 35 bp, Artificial Sequence
  constructing oligonucleotide
aaggtaaacgtgctaactactcttgcttctcttgc

SEQ ID NO: 34 - DNA, 31 bp, Artificial Sequence
  constructing oligonucleotide
tctaaaatcgctaacatgaccaacgaagaac

SEQ ID NO: 35 - DNA, 27 bp, Artificial Sequence
  constructing oligonucleotide
gtcgttcttcttgcccgttcgttggtg

SEQ ID NO: 36 - DNA, 27 bp, Artificial Sequence
  constructing oligonucleotide
acccggaacacatccgtgaacgtatca

SEQ ID NO: 37 - DNA, 27 bp, Artificial Sequence
  constructing oligonucleotide
aagacgttggtgctgacatcgaagaca

SEQ ID NO: 38 - DNA, 32 bp, Artificial Sequence
  constructing oligonucleotide
tcatgggtgaaggtccgttcaacggtaaatgc

SEQ ID NO: 39 - DNA, 27 bp, Artificial Sequence
  constructing oligonucleotide
acccgtctgctggaaaaactgacctgc

SEQ ID NO: 40 - DNA, 27 bp, Artificial Sequence
  constructing oligonucleotide
cgtaaacagtctcgtctggttgctacc

SEQ ID NO: 41 - DNA, 30 bp, Artificial Sequence
  constructing oligonucleotide
ccggttcgttactacctggaatgcaaagac

SEQ ID NO: 42 - DNA, 27 bp, Artificial Sequence
  constructing oligonucleotide
ccggaacgtaacggtggtgaaatctaa

SEQ ID NO: 43 - DNA, 18 bp, Artificial Sequence
  constructing oligonucleotide
ggttttagcacgtttcat

SEQ ID NO: 44 - DNA, 30 bp, Artificial Sequence
  constructing oligonucleotide
ggtgatacccggccaaccaccttctttagc

SEQ ID NO: 45 - DNA, 35 bp, Artificial Sequence
  constructing oligonucleotide
tagtcaacagaagatttagacagagcggtgtagaa

SEQ ID NO: 46 - DNA, 26 bp, Artificial Sequence
  constructing oligonucleotide
gttcagcgcatttacggaaggtttcg

SEQ ID NO: 47 - DNA, 33 bp, Artificial Sequence
  constructing oligonucleotide
caccttcttcgcaacgcagcatttcgatacgac

SEQ ID NO: 48 - DNA, 34 bp, Artificial Sequence
  constructing oligonucleotide
atagaggtagacaggcatttagagatgataccgt

SEQ ID NO: 49 - DNA, 29 bp, Artificial Sequence
  constructing oligonucleotide
agaagaagtgagacaggatgtcttcttcg

SEQ ID NO: 50 - DNA, 26 bp, Artificial Sequence
  constructing oligonucleotide
agcgtcctgagagcaaacaacacggc

SEQ ID NO: 51 - DNA, 36 bp, Artificial Sequence
  constructing oligonucleotide
gttaacttcagcgttaacgaaccaaacagcgttcca

SEQ ID NO: 52 - DNA, 28 bp, Artificial Sequence
  constructing oligonucleotide
ggtttttagagatacgcagacgcagcag

SEQ ID NO: 53 - DNA, 26 bp, Artificial Sequence
  constructing oligonucleotide
caggaagaaagcacgagcaccacccg

SEQ ID NO: 54 - DNA, 27 bp, Artificial Sequence
  constructing oligonucleotide
accgtccatgtgcggcagaacttcaga

SEQ ID NO: 55 - DNA, 25 bp, Artificial Sequence
  constructing oligonucleotide
ccagaacaccgtcctgggtacgagc

SEQ ID NO: 56 - DNA, 38 bp, Artificial Sequence
  constructing oligonucleotide
aacaacgtccgggttgtaacgagaacgcatacccagaa

SEQ ID NO: 57 - DNA, 30 bp, Artificial Sequence
  constructing oligonucleotide
aaccagaacttcagaagaagcagcagaagc

SEQ ID NO: 58 - DNA, 28 bp, Artificial Sequence
  constructing oligonucleotide
cgatcaggtcgataactttggtgaagtg

SEQ ID NO: 59 - DNA, 31 bp, Artificial Sequence
  constructing oligonucleotide
taaccaccttccggaacaacagcacgtttac

SEQ ID NO: 60 - DNA, 33 bp, Artificial Sequence
  constructing oligonucleotide
agagaaccgataccttcgtcgttcagacgcagg

SEQ ID NO: 61 - DNA, 30 bp, Artificial Sequence
  constructing oligonucleotide
gacaggtaacgacggaattcagaaaccagc

SEQ ID NO: 62 - DNA, 36 bp, Artificial Sequence
  constructing oligonucleotide
ttagaagagatttcaaccagttcgtccatacggtta

SEQ ID NO: 63 - DNA, 27 bp, Artificial Sequence
  constructing oligonucleotide
agaacacgcatacgttcgtccggttcg

SEQ ID NO: 64 - DNA, 25 bp, Artificial Sequence
  constructing oligonucleotide
cgggttagccagcagagaggtagac

SEQ ID NO: 65 - DNA, 32 bp, Artificial Sequence
  constructing oligonucleotide
agagagaagttggtaccacgttcgtaaccggt

SEQ ID NO: 66 - DNA, 29 bp, Artificial Sequence
  constructing oligonucleotide
acggcgggaagtaacgttcagaagattcc

SEQ ID NO: 67 - DNA, 25 bp, Artificial Sequence
  constructing oligonucleotide
agacgttccatgatgtcctgcatgc

SEQ ID NO: 68 - DNA, 32 bp, Artificial Sequence
  constructing oligonucleotide
ggtcgttgtatttcaggtgtttgtttttacgc

SEQ ID NO: 69 - DNA, 34 bp, Artificial Sequence
  constructing oligonucleotide
ccgcagtctttcaggaacaggcacagggtctgac

SEQ ID NO: 70 - DNA, 31 bp, Artificial Sequence
  constructing oligonucleotide
gaagaaagcgataccgtcttcaacagacata

SEQ ID NO: 71 - DNA, 28 bp, Artificial Sequence
  constructing oligonucleotide
cttcacgcggagctttgaaagaaccacg

SEQ ID NO: 72 - DNA, 35 bp, Artificial Sequence
  constructing oligonucleotide
gtgacggatagagtacaggtattctttgtcgaaaa

SEQ ID NO: 73 - DNA, 27 bp, Artificial Sequence
  constructing oligonucleotide
agcacgtttaccttccagaccgtagtt

SEQ ID NO: 74 - DNA, 38 bp, Artificial Sequence
  constructing oligonucleotide
atgttagcgattttagagcaagagaagcaagagtagtt

SEQ ID NO: 75 - DNA, 29 bp, Artificial Sequence
  constructing oligonucleotide
ggcaagaagaacgacgttcttcgttggtc

SEQ ID NO: 76 - DNA, 23 bp, Artificial Sequence
  constructing oligonucleotide
gtgttccgggtcaccaacgaacg

SEQ ID NO: 77 - DNA, 29 bp, Artificial Sequence
  constructing oligonucleotide
gcaccaacgtctttgatacgttcacggat

SEQ ID NO: 78 - DNA, 31 bp, Artificial Sequence
  constructing oligonucleotide
cggaccttcacccatgatgtcttcgatgtca

SEQ ID NO: 79 - DNA, 26 bp, Artificial Sequence
  constructing oligonucleotide
agcagacgggtgcatttaccgttgaa

SEQ ID NO: 80 - DNA, 30 bp, Artificial Sequence
  constructing oligonucleotide
cgagactgtttacggcaggtcagtttttcc

SEQ ID NO: 81 - DNA, 29 bp, Artificial Sequence
  constructing oligonucleotide
ggtagtaacgaaccggggtagcaaccaga

SEQ ID NO: 82 - DNA, 41 bp, Artificial Sequence
  constructing oligonucleotide
ttagatttcaccaccgttacgttccgggtctttgcattcca

(19) United States
(12) Patent Application Publication
(10) Pub. No.: US 2012/0156731 A1
(43) Pub. Date: Jun. 21, 2012

(54) Improved Methods for Rapid Gene Synthesis

(75) Inventors: Yanyi Huang, Beijing (CN); James Berger, Kensington, CA (US); Stephen Quake, Stanford, CA (US)

(73) Assignee: The Board of Trustees of the Leland Stanford Junior University, Palo Alto, CA (US)

(21) Appl. No.: 12/052,156

(22) Filed: Mar. 20, 2008

Related U.S. Application Data
(60) Provisional application No. 60/919,661, filed on Mar. 23, 2007.

(51) Int. Cl. C12P 19/34; C07H 21/04; C12M 1/00; C12N 9/00

(52) U.S. Cl. 435/91.52; 435/183; 536/23.1; 435/289.1

(57) ABSTRACT

Disclosed are methods and materials for assembling long polynucleotides from synthetic oligonucleotides. The use of synthetic oligonucleotides permits non-natural design of sequences. The oligonucleotides used for construction may be relatively short, according to practicalities of nucleotide synthesis. They are assembled using a ligase which is operative over a range of temperatures, i.e., is thermostable. The method and oligonucleotides are designed such that the melting temperature of the strands to be hybridized is set at a number of selected specific temperatures for each group of oligonucleotides to be hybridized and ligated. Hybridization and ligation take place at or near the melting temperature, so that each succeeding ligation is governed by a temperature that will prevent hybridization if any mismatches are present.

### CROSS-REFERENCE TO RELATED APPLICATIONS

This application claims priority from U.S. Provisional Patent Application No. 60/919,661 filed on Mar. 23, 2007, which is hereby incorporated by reference in its entirety.

### STATEMENT OF GOVERNMENTAL SUPPORT

This invention was made with U.S. Government support under DARPA Grant Number HR0011-04-1-0032. The U.S. Government has certain rights in this invention.

### REFERENCE TO SEQUENCE LISTING, COMPUTER PROGRAM, OR COMPACT DISK

Applicants assert that the paper copy of the Sequence Listing is identical to the Sequence Listing in computer readable form found on the accompanying computer disk. Applicants incorporate the contents of the sequence listing by reference in its entirety.

### BACKGROUND OF THE INVENTION

1. Field of the Invention

The present invention relates to the field of assembly of polynucleotides from oligonucleotides.

2. Related Art

Synthetic genes of designed sequences are assembled one at a time by two major methods: (1) assembly polymerase chain reaction (PCR), or (2) ligation reaction of smaller oligonucleotides that have overlapping homologies.

The PCR assembly method is based on the assembly of oligonucleotides, and the use of DNA polymerase to synthesize DNA on a template. In the first step of assembly PCR, multiple oligodeoxynucleotides that contain overlapping regions anneal, and a DNA polymerase extends the primers and fills in the regions between the primers. A range of products of different lengths results from the different possible combinations of annealing that involve less than all the oligodeoxynucleotides. In the second PCR step a pair of primers is introduced that is specific for the full-length oligodeoxynucleotide and the full-length product is selectively amplified from the mixture. See, Rydzanicz, “Assembly PCR oligo maker: a tool for designing oligodeoxynucleotides for constructing long DNA molecules for RNA production,” Nucleic Acids Research, Volume 33, Web Server Issue Pp. W521-W525. This paper describes the creation of a computer program in Java for designing oligonucleotides from a given end product DNA sequence. In this program, all oligos necessary for a two-step synthesis are designed to have a uniform melt temperature for a single step assembly of multiple oligos. A 191-nt long DNA molecule was created using the sequences suggested by the program.

Gene synthesis by ligation is carried out by ligating a number of smaller (20-80 bases in length) oligonucleotides that contain overlapping homologies. Occasionally, gaps will be built into each complementary strand, and a DNA polymerase will be used in conjunction with a DNA ligase to fill the gap and covalently link the fragments. As is known in this process, once an area of partial or incomplete homology is lengthened by ligation, the thermal stability of the DNA duplex is greatly enhanced, resulting in the increased likelihood of synthesizing DNA with an incorrect sequence. Higher temperatures reduce the occurrence of these temporary hybridizations, although the low optimal reaction temperatures of standard DNA ligases such as T4 DNA Ligase (15-22° C.) limit the success of this approach. A method has been published by Epicentre Biotechnologies (www.epibio.com) using thermostable Ampligase DNA ligase, where the oligonucleotides are incubated at a succession of temperatures, starting at 60° C. for one hour, then 50° C., 40° C., 30° C. and 20° C. in a one-tube procedure. This procedure was designed to produce a 380 bp gene from 18 oligonucleotides of 40-50 bases in length with 10-20 base overlaps. The protocol does not suggest whether or when to use different overlap lengths.

Both of these existing methods are susceptible to errors in the assemblies, which errors will propagate to the final product.

In the conventional assembly PCR gene synthesis method, synthetic errors (deletions, insertions, or mutations) in any of the constructing oligonucleotide strands will be transferred into the final gene product. In the final PCR amplification step, the errors will be copied as well. To reduce this error rate, a purification step for each of the constructing oligonucleotides is necessary, which drastically increases the cost of the gene synthesis.

Ligation-based gene synthesis methods are less susceptible to the oligonucleotide error rate, since the ligation process is sensitive to deletions and insertions. However, the strands with synthetic errors will still have the possibility of hybridizing with other strands and the error will transfer to the final full-length gene product. Additionally, assembly efficiency can be reduced by mis-hybridization due to the large number of sequences that must hybridize at the same temperature.

### SPECIFIC PATENTS AND PUBLICATIONS

Jayaraman et al., “Polymerase Chain Reaction-Mediated Gene Synthesis: Synthesis of a Gene Coding for Isozyme c of Horseradish Peroxidase,” Proc. Nat. Acad. Sci., 1991, Vol 88, 4084-4088, report on a process where all the oligonucleotides making up the gene to be synthesized are ligated in a single step by using the two outer oligonucleotides as PCR primers and the crude ligation mixture as the target. It is reported that the size of the PCR products obtained from a single-step ligation can be increased by increasing the length of individual oligonucleotides (>100-mers) without increasing the number of oligonucleotides or by increasing both the number and the length of oligonucleotides. This is a strategy whereby gene fragments are first generated by PCR and then joined together in-frame.

Tian, H. Gong, N. Sheng, X. Zhou, E. Gulari, X. Gao, G. Church, “Accurate Multiplex Gene Synthesis from Programmable DNA Chips,” Nature, 2004, 432, 1050-1054 discloses synthesis of DNA oligos on a chip followed by a PCR-assembly method. The authors used a “ligation-selection” method to reduce the error rate. (The error rate is 1 error per 1394 bp). Pools of thousands of “construction” oligonucleotides and tagged complementary “selection” oligonucleotides are synthesized on a chip, released, amplified and selected by hybridization.

G. Chen, I. Choi, B. Ramachandran, J. E. Gouaux, “Total gene synthesis: novel single-step and convergent strategies applied to the construction of a 779 base pair bacteriorhodopsin gene,” J. Am. Chem. Soc., 1994, 116, 8799-8800, describes the PCR assembly of 12 oligos into a 779 bp gene. Long oligos having unique overlaps of about 20 bp in length were designed. The oligos were between 70 and 100 nucleotides in length. The lengths of the short oligos were selected to allow annealing at approximately 50° C.

W. P. C. Stemmer, A. Crameri, K. D. Ha, T. M. Brennan, H. L. Heyneker, “Single-step assembly of a gene and entire plasmid from large numbers of oligodeoxyribonucleotides,” Gene, 1995, 164, 49-53, discloses assembly PCR as a method for the synthesis of long DNA sequences from large numbers of oligodeoxyribonucleotides. The method does not rely on DNA ligase but instead relies on DNA polymerase to build increasingly longer DNA fragments during the assembly process The authors used 56 40-mers or 134 40-mers to synthesize 2 different genes, 0.9 kb and 2.7 kb, respectively.

K. E. Richmond, et al, “Amplification and assembly of chip-eluted DNA (AACED): a method for high-throughput gene synthesis,” Nucleic Acid Research, 2004, 32, 5011-5018 describe a method based on the photolithographic synthesis of long (>60 mers) single-stranded oligonucleotides, using a modified maskless array synthesizer. Once the covalent bond between the DNA and the glass surface is cleaved, the full-length oligonucleotides are selected and amplified using PCR. Subsequent gene assembly experiments using this DNA pool were performed and were successful in creating longer DNA fragments.

P. A. Carr, J. S. Park, Y. Lee, T. Yu, S. Zhang, J. M. Jacobson, “Protein-mediated error correction for de novo DNA synthesis,” Nucleic Acid Research, 2004, 32, e162, employ a DNA mismatch-binding protein, MutS (from Thermus aquaticus) to remove failure products from synthetic genes, reducing errors by >15-fold.

B. F. Binkowski, K. E. Richmond, J. Kaysen, M. R. Sussman, P. J. Belshaw, “Correcting errors in synthetic DNA through consensus shuffling,” Nucleic Acid Research, 2005, 33, e55 also used MutS to get ˜1 error per 3500 bp.

X. Zhou, S. Cai, A. Hong, Q. You, P. Yu, N. Sheng, O, Srivannavit, S. Muranjan, J. M. Rouillard, Y. Xia, X. Zhang, Q. Xiang, R. Ganesh, Q. Zhu, A. Matejko, E. Gulari, X. Gao, “Microfluidic picoarray synthesis of oligodeoxynucleotides and simultaneous assembling of multiple DNA sequences,” Nucleic Acids Research, 2004, 32, 5409-5417 reports on the use of Taq Ligase to make a 714 bp EGFP gene and 712 bp EYFP gene. The oligonucleotides were on average, 30 or 45 mer fragments with cohesive joints. The ligation products were divided into several portions and each was PCR-amplified with a high-fidelity polymerase (PfuUltra, Stratagene) using several primer pairs specific for amplifying different regions of the ligated sequence.

U.S. Pat. No. 6,110,668 to Strizhov, et al., issued Aug. 29, 2000, entitled “Gene synthesis method,” discloses a method that utilizes a combination of enzymatic and chemical synthesis of DNA. In this method, chemically synthesized and phosphorylated oligonucleotides of the gene to be created are assembled on a single-stranded partially homologous template DNA derived from the natural or wild-type gene. After annealing, the nicks between adjacent oligonucleotides are closed by a thermostable DNA ligase using repeated cycles of melting, annealing, and ligation. This template directed ligation (“TDL”) results in a new single-stranded synthetic DNA product which is subsequently amplified and isolated from the wild type template strand by the polymerase chain reaction (PCR) with short flanking primers that are complementary only to the new synthetic strand.

### BRIEF SUMMARY OF THE INVENTION

The following brief summary is not intended to include all features and aspects of the present invention, nor does it imply that the invention must include all features and aspects discussed in this summary.

Described here is an improved method, based on multistage ligation reactions, to reduce the error rate of gene synthesis using unpurified chemically synthesized oligonucleotides.

The present invention comprises a method for assembling a double stranded polynucleic acid molecule of any defined sequence from a plurality of single stranded oligonucleotides. The assembled polynucleotide may be of any length. The method does not rely on polymerase incorporation of individual nucleotides. Rather, it comprises the steps of preparing a set S of oligonucleotides having 5′ end portions (5′E) and 3′ end portions (3′E). That is, each oligo is designed to have end portions, which hybridize to another synthetic oligo under specific hybridization conditions (normally determined by temperature). Each oligonucleotide in set S comprises 5′E and 3′E portions having at least one sequence complementary to another oligonucleotide in set S, where said set S together is sufficient to construct the final long polynucleic acid. By using a specific complementary sequence for each oligonucleotide one obtains a plurality of different, discrete melting temperatures, which effectively divides the total set S of oligonucleotides into subsets S₁ through Sₙ each with a different melting point between the two complementary sequences. One then sequentially combines and ligates subsets S₁ through Sₙ at a different temperature for each subset until said polynucleic acid molecule is assembled.

Thus, the method may be said to comprise an improved method of synthesis by ligation of partially overlapping oligonucleotides, where the oligonucleotides have been designed to comprise a complete set S, whereby no polymerase is needed. The set S is divided into subsets, e.g. about 2-5 subsets, where each subset can contain pools of 2-3 oligonucleotides designed to specifically hybridize to each other. Each subset will hybridize at a discrete, different temperature. A thermostable ligase is used to ligate the partially overlapping, overhanging oligonucleotides. The method for assembling a double stranded polynucleic acid molecule of a defined sequence from a set S of single stranded oligonucleotides thus comprises the steps of: preparing a set S of oligonucleotides having 5′ end portions (hereafter 5′E) and 3′ end portions (hereafter 3′E), wherein at least one of said 5′E and 3′E portions have a complementary sequence to another oligonucleotide in set S, said complementary sequence for each oligonucleotide comprised of a plurality of different sequences having different, discrete melting temperatures and thereby dividing set S into subsets S1 through Sn each with a different, discrete melting point as between the end portions; and c) sequentially combining and ligating oligonucleotides from subsets S1 through Sn at a different temperature for each subset until said polynucleotide molecule is assembled into the full length double stranded polynucleic acid molecule from said oligonucleotides which are successively hybridized at said 5′E and 3′E portions at successive steps at different, elevated temperatures, and ligated at successive steps at an elevated temperature.

Specific hybridization takes place among the oligonucleotides in a given subset. This hybridization takes place under specific conditions, namely at or near a specific temperature at which no mismatched nucleotides will hybridize. Alternatively the temperature protocol may be designed around the overhang length and sequence. That is, different lengths of overhang will hybridize at different temperatures, permitting a single pool containing all subsets S₁ through Sₙ to be hybridized over a changing temperature gradient. It is understood that some naturally occurring (rather than synthetic) duplexes may also be incorporated into this method, and their 5′ or 3′ overhangs created by selecting appropriate restriction enzymes. For ease of design, each subset may be thought of as comprising a multiple of three oligos, in that three oligos will be combined to form a duplex with overhangs to be used in the next step, with a ligation point between two adjacent oligos.

In certain aspects of the invention, the polynucleic acid molecule to be constructed is DNA, such as an artificial gene. Modified bases or sugars may be incorporated to, e.g., prevent enzymatic degradation, or to study various genetic effects. Non-natural nucleotides may be included in the synthetic oligonucleotides, which can be chemically synthesized by a number of known methods (see U.S. Pat. No. 5,541,307). RNA may be constructed in hybrid duplexes or in ds RNA complexes, using RNA ligase.

In one aspect, the invention comprises the use of oligonucleotides in set S, which are, prior to any ligation, between about 20 and 100 nucleotides in length. Due to the desirability of accurate synthesis (<1 error per 1000 nt, without a repair step), it is preferable to have oligonucleotides in set S that comprise at least 80 oligonucleotides prior to ligation. Typically succeeding subsets will be built up through several rounds of ligation and hybridization. That is, the duplexes from subset S₁ will themselves be combined in subset S₂, resulting in longer duplexes, which will be combined in subset S₃, etc., until the entire set S is used to prepare the final polynucleotide. In other words, the set of oligonucleotides may comprise a number of oligonucleotides equal to about 1/30 of the number of nucleotides in the final polynucleic acid, assuming that each oligonucleotide is about 30 nt long. As understood in the art, an oligonucleotide for synthesis by ligation may be about 20-80 nt in length. For example, an oligonucleotide 30 nt in length may have a 15 nt 5′E and a 15 nt 3′E overlap region, allowing for ligation.

In one aspect, the invention comprises the use of a ligase that is thermostable at various preselected temperatures. According to the present invention, one may use preselected temperatures for each subset S, differing by at least 2° C. and between 20° C. and 55° C., and/or in a range of 50 to 65°. In one aspect of the invention, the temperature steps between subsets S₁, S₂, etc. increase so as to prevent mismatched hybridization from proceeding. Thus, each temperature step may vary by between about 4 and 6° C. In order to avoid the use of individual nucleotides, the overlapping end portions 5′E and 3′E may be immediately adjacent in the final polynucleic sequence (i.e., there is no nucleotide in between them). In this case, no individual nucleotides are added and no polymerase is added.

In another aspect, the present invention comprises a set S of oligonucleotides for assembling a double stranded polynucleic acid molecule of a defined sequence. The set S may contain a plurality of single stranded pre-prepared oligonucleotides, where each oligonucleotide has a 5′ end portion (5′E) and a 3′ end portion (3′E) that overlaps with one or two other oligonucleotides in set S. The 5′E and 3′E portions preferably have a sequence and length resulting in a plurality of different, discrete melting temperatures, thereby dividing set S into subsets S₁ through Sₙ. Preferably, n is at least 3. The number of oligonucleotides used to make up set S can vary widely, and may be designed as triplets where each oligo hybridizes at its 3′ and 5′ end to the 5′ and 3′ ends respectively of two other oligos. Thus, in certain aspects, the invention comprises the use of 3′ oligonucleotides, where X is between 1 and 10, and is preferably at least 3. In other words, the overhanging duplex (as shown in FIG. 1A) is regarded, prior to ligation, as three oligonucleotides. There is overhang at the 3′ and the 5′ end of the oligos, which are to be ligated. It is also contemplated by the present invention that the overlap region between an oligo and its hybridization partner may be between 5 and 25 nucleotides.

The present methods may be implemented in kit form, as is known in the art, whereby one provides a combination which may include software, thermostable ligase enzyme, buffers, instructions for use and/or a thermocycler. The user will obtain oligos based on the desired synthetic product.

In one aspect, the present invention comprises a computer program for designing a plurality of oligonucleotides which, when assembled, form a user predefined polynucleic acid molecule of sequence SEQ from the plurality of single stranded oligonucleotides. The program determines a set S of oligonucleotides having length L and 5′ end portions (5′E) and 3′ end portions (3′E) overlapping with another oligonucleotide in set S. The 5′E and 3′E portions of each oligonucleotide are designed to have a sequence and length resulting in a plurality P of different, discrete melting temperatures, thereby dividing set S into subsets S₁ through Sₙ. Each subset is designed to contain at least three oligonucleotides prior to ligation, such that sequentially combining and ligating subsets S₁ through Sₙ at a different temperature for each subset results in assembly of said polynucleic acid molecule. Each subset is further designed to have a higher melting temperature, thereby eliminating mismatches.

It should also be noted that the present synthetic method yields a polynucleic acid molecule that can be used directly, without further processing or purification. For example, the DNA may be cloned directly into a vector.

### BRIEF DESCRIPTION OF THE DRAWINGS

FIGS. 1A and B shows a simplified schematic illustration of the design of DNA fragments according to the present methods;

FIGS. 2A and B is another schematic illustration of the assembly of DNA fragments according to the present invention;

FIG. 3 is a schematic illustration of multistage ligation synthesis with a synthetic error in an oligonucleotide; and

FIG. 4 is a photograph of agarose gel electrophoresis of a DNA molecule made according to the present method.

### DETAILED DESCRIPTION OF THE PREFERRED EMBODIMENT

### Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference. The heading provided herein are for convenience only and do not limit the invention in any way.

As used herein, “a” or “an” means “at least one” or “one or more.”

The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. The term “nucleotide” is also used herein to encompass “modified nucleotides” which comprise at least one modification: (a) an alternative linking group, (b) an analogous form of purine, (c) an analogous form of pyrimidine, or (d) an analogous sugar, all as described herein. “Analogous” forms of purines and pyrimidines are those generally known in the art, many of which are used as chemotherapeutic agents. An exemplary but not exhaustive list includes aziridinylcytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyl-uracil, 5-methoxyaminomethyl-2-thiouracil, beta-D-mannosylqueosine, 5-methoxyuracil, 2-methyl-thio-N6-isopentenyladenine, uracil-5-oxyacetic acid methylester, pseudouracil, queosine, 2-thiocytosine, 5-methyl-2-thiouracil, 2-thiouracil, 4-thiouracil, 5-methyluracil, uracil-5-oxyacetic acid, 5-pentynyluracil and 2,6-diaminopurine. The use of uracil as a substitute base for thymine in deoxyribonucleic acid (hereinafter referred to as “dU”) is considered to be an “analogous” form of pyrimidine in this invention. Additional examples of artificial bases useful as nucleotides are found in U.S. Pat. No. 5,126,439 to Rappaport, issued Jun. 30, 1992, entitled “Artificial DNA base pair analogues.”

The oligonucleotides of the long polynucleotides of the invention may contain analogous forms of ribose or deoxyribose sugars that are generally known in the art. An exemplary, but not exhaustive list includes 2′ substituted sugars such as 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and basic nucleoside analogs such as methyl riboside.

Although the conventional sugars and bases may be used in applying the methods of the invention, substitution of analogous forms of sugars, purines and pyrimidines can be advantageous in designing the final product, as can alternative backbone structures like a polyamide backbone.

In addition the polynucleotides of the invention may be comprised of short (the equivalent of up to 3 nucleotides in length) “spacer compounds” which duplicate the length and spatial geometry of a nucleotide, but do not engage in Watson-Crick-type binding with other nucleotides, and are not subject to cleavage by endonucleases. However, the polynucleotides of the invention are preferably comprised of greater than 50% conventional deoxyribose nucleotides, and most preferably greater than 90% conventional deoxyribose nucleotides.

In the above-described instances, one may determine melting temperatures empirically if the non-natural nucleotides are in overlapping regions. They may also be incorporated into non-hybridizing regions and filled in with dNTP and polymerase.

The term “melting temperature”, denoted “Tm”, means the midpoint of the duplex-to-single-strand melting transition of a duplex nucleic acid, that is, half of the duplex will be hybridized and half will be single stranded, on average. The Tm of a duplex can be measured by methods well known in the art, some of which are described below. The Tm of double-stranded DNA (dsDNA) refers to a temperature at which 50% of a dsDNA sample is separated into its two complementary DNA strands. The term “discrete melting temperatures” means melting temperatures that are a specific temperature plus or minus less than about 3%, and wherein each temperature is distinct from the other. For example 60° C., ±1.8° C., and 65° C., ±2° C. are two discrete melting temperatures.

The term “ligating” means covalently attaching polynucleotide sequences together to form a single sequence. This is typically performed by treatment with a ligase, which catalyzes the formation of a phosphodiester bond between the 5′ end of one sequence and the 3′ end of the other. However, in the context of the invention, the term “ligating” is also intended to encompass other methods of covalently attaching such sequences, e.g., by chemical means. The terms “covalently attaching” and “ligating” may be used interchangeably.

The term “ligase” means an enzyme that catalyzes the formation of a phosphodiester bond between adjacent 3′ hydroxyl and 5′ phosphoryl termini of oligonucleotides that are hydrogen bonded to a complementary strand and the reaction is termed “ligation”.

The term “thermostable” is used in connection with a ligase which maintains its activity at a temperature above at least about 37° C. for at least an hour. Thermostable DNA ligases, per se, are well known in the art and are commercially available. For example, a thermostable DNA ligase from Pyrococcus furiosus (Pfu DNA ligase; U.S. Pat. Nos. 5,506,137 and 5,700,672, hereby incorporated by reference) is available from Stratagene (La Jolla, Calif.). This enzyme catalyzes the linkage of adjacent 5′-phosphate and 3′-hydroxy ends of double-stranded DNA at about 45° C. to 80° C. The enzyme is highly thermostable, having a half-life of greater than 60 minutes at 95° C., and the temperature optimum for nick-sealing reactions is about 70° C. By way of further example, Taq DNA ligase (from Thermus aquaticus) catalyzes the formation of a phosphodiester bond between juxtaposed 5′-phosphate and 3′-hydroxyl termini of two adjacent oligonucleotides that are hybrized to a complementary DNA. Taq DNA ligase is active at elevated temperatures (45° C. to 65° C.). F. Barany, 88 Proc. Nat'l Acad. Sci. USA, 1991, 189; M. Takahashi et al., 259 J. Biol. Chem., 1984, 10041-10047. By way of still further example, AMPLIGASE® thermostable DNA ligase (Epicentre Technologies) catalyzes NAD-dependent ligation of adjacent 5′-phosphorylated and 3′-hydroxylated termini in duplex DNA structures. This enzyme has a half-life of 48 hours at 65° C. and greater than 1 hour at 95° C. This thermostable DNA ligase has also been shown to be active for at least 500 thermal cycles (94° C./80° C.) or 16 hours of cycling. M. Schalling et al., 4 Nature Genetics, 1993, 135.

As another example, Blondal et al., “Isolation and characterization of a thermostable RNA ligase 1 from a Thermus scotoductus bacteriophage TS2126 with good single-stranded DNA ligation properties,” Nucleic Acids Research, 2005, 33(1):135-142, discloses a thermostable RNA ligase. RNA ligases have the ability to ligate single-stranded nucleic acids by catalyzing the ATP-dependent formation of phosphodiester bonds between 5′-phosphate and 3′-hydroxyl termini of single-stranded RNA or DNA.

“Hybridization” refers to the process by which a polynucleotide strand anneals with a complementary strand through base pairing under defined hybridization conditions. Specific hybridization is an indication that two nucleic acid sequences share a high degree of identity. Specific hybridization complexes form under permissive annealing conditions and remain hybridized after various steps that may cause separation, commonly termed “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml denatured salmon sperm DNA.

Generally, stringency of hybridization is expressed, in part, with reference to the temperature under which the wash step is carried out. Generally, such wash temperatures are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook et al., 1989, Molecular Cloning: A Laboratory Manual, 2^{nd} ed., vol. 1 3, Cold Spring Harbor Press, Plainview N.Y.; specifically see volume 2, chapter 9. As described below, a primer that does not entirely match the target is used with appropriate stringency. The “stringency” here is achieved by varying the temperature, magnesium concentration, or both, in the annealing steps where primer and target bind to each other in PCR, or probe and target bind to each other in the SMART reaction. The important point here is that the annealing take place under the buffer conditions of the enzymatic reaction.

### Overview

The present invention introduces new methods of gene assembly that allow sequential assembly and error correction by thermal stepping and multi-pot reactions. By using either a variety of temperatures, or a variety of separate reaction vessels, or a combination thereof, one can dramatically improve the quality of gene synthesis. In particular, one can greatly reduce the effects of errors in the oligonucleotide precursors, even to the point of using unpurified material.

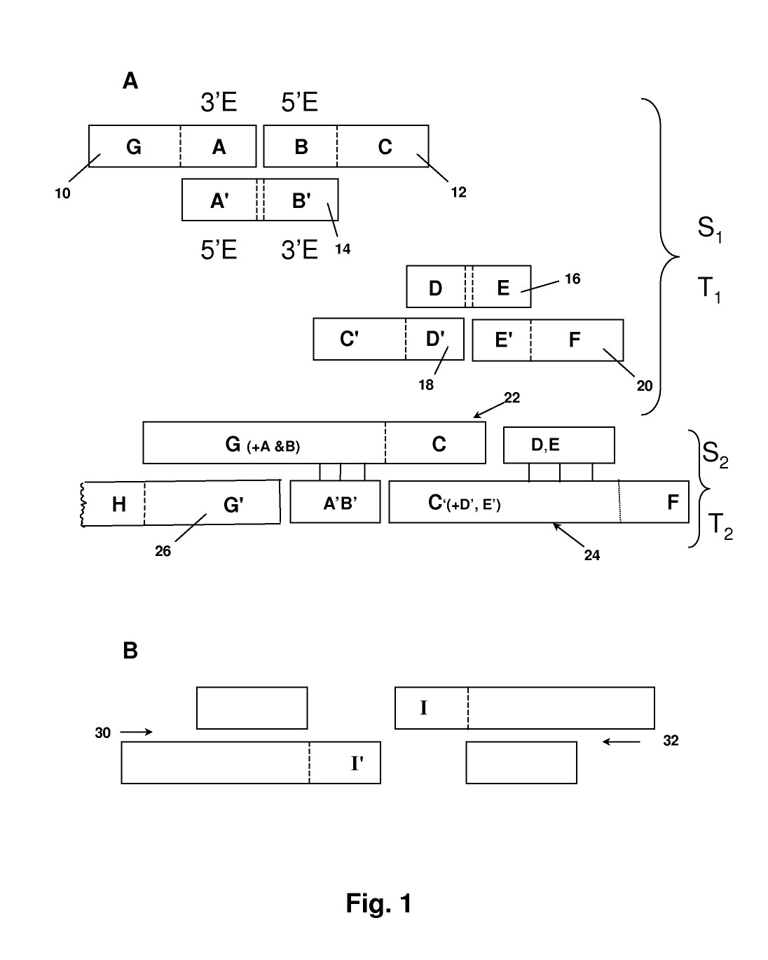

Referring now to FIG. 1, a schematic diagram illustrates a set S of oligonucleotides assembled according to the present methods, and their specifically determined overlap. In FIG. 1A, oligonucleotides 10 and 12 are to be ligated with the use of template oligo 14. They have template overlap regions A (3′E) and B (5′E) with sequences complementary to sequences A′ (5′E) and B′ (3′E) on oligo 14. When the three are mixed below the melting temperature, a duplex will form between the three oligos 10, 12, and 14 due to Watson Crick base pairing between A and A′ and B and B′. In the next step in the process, a ligase will be used to link A and B by forming a phosphodiester bond between them. This will add a bond between the bases opposite the dotted lines shown in the oligonucleotide 14, A′ B′. The same holds true for oligos 16, 18, and 20, which have overlap regions D and E (on oligo 16), C′ and D′, and E′ and F, respectively. D will hybridize to D′, and E to E′. These six oligos are designed to hybridize and be ligated in a first step, in the same mixture, at the same temperature T₁ as oligos 10, 12 and 16. Thus, oligos 10, 12, 14, 16, 18, and 20 are members of subset S₁. This step may include additional oligos, such as three (or more) different pools. They will all have the same approximate melting temperature T₁. In the next step, the duplexes formed in step 1, which are now the combined length of oligo 10 plus oligo 12 and the combined length of oligo 18 plus 20, are combined at temperature T₂ which is higher than T1, such that any mismatches from the previous step are melted. This is seen as the hybridizing of complex 22 (formerly oligos 10, 12 and 14) and complex 24 (formerly oligos 16, 18 and 20) in the previous step. Complexes 22 and 24 are hybridized with another complex 26 (partially illustrated) which results also from step 1 in a similar manner of hybridization of the oligos described in step 1. This complex is similarly designed to hybridize in a second step in overlap regions G′ and C′, which are of the same melting temperature T₂, but a different melting temperature than in the first step. Thus, oligos 22, 24 and 26 are members of set S₂. In each case, hybridization is carried out near the melting temperature (i.e., under stringent conditions) to prevent mismatches. Then, in a third step, not shown, hybridization will occur at F and H. As many subsequent steps as desired can be created, each with a different hybridization overlap region melting temperature. That is, each synthetic step is carried out at a specific condition of salt concentration, base content (% G+C) in the overlap region, and temperature, so that annealing can only take place if there is a 100% match. Each succeeding step has a different condition, most conveniently temperature, for controlled annealing, and any number of steps may be employed within the temperature range and steps available.

As shown here in FIG. 1, each succeeding overlap region is longer, and has a higher melting temperature. However, a process can be designed so that each succeeding step can be done at a lower temperature. In this case, the oligos are all mixed into a single reaction mixture, and the temperature is lowered through different hybridization/melting temperatures.

FIG. 1B, as discussed below, shows an alternative embodiment in which oligos are hybridized for overlap of I and I′, where the desired overhangs are created by restriction enzymes which produce sticky ends.

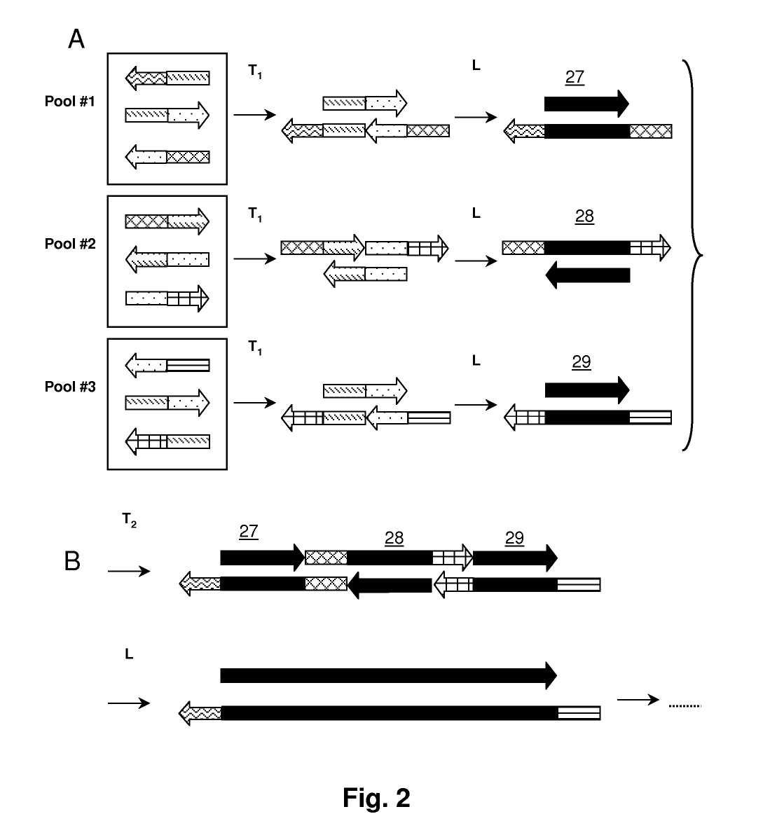

FIG. 2 shows the schematic procedure of one example of this method. In a first stage, shown in FIG. 2A, three pools, each composed of three different oligos (9 different oligos total), are created, mixed together, and ligated to form duplexes 27, 28, and 29. In the first, conceptual step, a set S of oligonucleotides is designed from the full-length polynucleotide sequence that is desired. In other words, one would work from the end of the process of FIG. 2 upwards. In order to design the oligos, the full-length polynucleotide with a specific sequence is divided into small fragments (constructing oligonucleotides) by a computer program. Each piece has two sections, and the length of each section is designed by optimizing the melting temperature to fit a certain reaction temperature (see below). For example, in FIG. 2, each oligonucleotide (which has been 5′ phosphorylated) is shown as an arrow with two sections (illustrated with different fill patterns). The direction of arrow indicates the DNA sequence from the 5′-end to the 3′-end. In the first step, three oligonucleotides are combined together, shown as a “pool” in the figure. The three oligonucleotides are designed to have complementary sequences (different fill patterns indicate different complementary sections in the oligonucleotides; the same fill pattern from different strands means these two strands will hybridize together due to the complementary sequences) to each other so that after mixing they will form a hybridized complex. In this case, three pools are formed. Next, the three oligos in each pool are mixed together at temperature T₁. This step is illustrated in FIG. 2A as arrows labeled T₁. The mixing temperature T₁ is controlled to be a temperature that is close to the designed melting temperature of the complement annealing strands in this step. Then the thermo-stable ligase L is introduced into the mixer and the mixer is incubated at T₁ to complete the reaction. This step is illustrated in FIG. 2A as arrows labeled L. The ligase will form the covalent phosphodiester bonds between the 5′ phosphate end of one oligonucleotide and the 3′ hydroxyl group of the other one when these two oligonucleotides are brought together by hybridization with another strand with sequence complementary to both of them. In the figure, black color is used to indicate the strands after ligation.

In the second stage, shown in FIG. 2B, the three products of the first stage (from 9 oligonucleotides in the beginning) are mixed together at a higher temperature T₂, which is close to the melting temperature of the complementary strands in this step. This step is illustrated in FIG. 2B as arrows labeled T₂. The three complexes will form a bigger complex due to the hybridization of complementary strands. Next, ligase will join the hybridized fragment as in the previous stage and form a larger double strand DNA. This step is illustrated in FIG. 2B as arrows labeled L.

By repeating the procedure (with increased reaction temperature), at every stage, three smaller DNA pieces will be combined into a bigger one, with one strand having overhanging 5′ and 3′ ends for further joining (up until the last step).

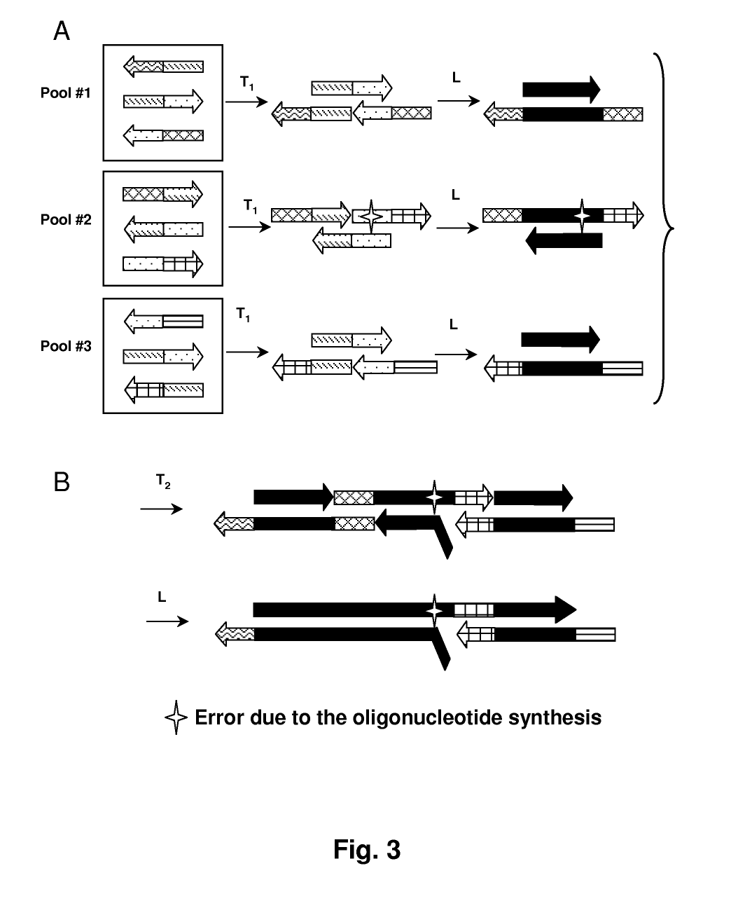

In the present method, using a multistage strategy with an increased ligation temperature at each stage, one can reduce the error rate of gene (polynucleotide) synthesis. FIG. 3 illustrates a case in which there is a synthetic error (deletion, insertion, or mutation) in one of the constructing oligonucleotide strands. If in the first stage, shown in FIG. 3A, the error doesn't interfere, the ligation reaction and the complex will still form. The error will be transferred to the second stage (shown in FIG. 3B). But, when the temperature is raised to T₂, the strand with error, which caused the false hybridization at T₁, will not be able to hybridize to the complement anymore due to the increased hybridization stringency. Hence the ligation will not be able to complete at this stage. Consequently, the final gene with right length will not contain the strands with error.

By using this multi-stage ligation method, constructing oligonucleotides can be used directly without further purification.

This method can also be implemented using a “one pot” synthesis. In this case, the oligonucleotides are designed with a difference temperature order. The first oligos to be assembled will have the highest melting temperature, so that they assemble and ligate while nothing else in the reaction can. Then when the temperature is lowered to the next step, the second set of oligos will ligate together the pieces that had been assembled at the higher temperature. This process is then repeated until the construct is finished.

Another implementation of the method is to design arbitrary melting temperatures (for example, constant temperatures for all ligations, or else temperatures chosen to optimize some parameter specific to each sequence), and to perform the assembly in separate reactions. After each step, the results of reactions are combined into a new reaction, at a different temperature, as appropriate. It is also possible to perform a “clean-up” step after each step in order to remove or neutralize unreacted product.

The present methods and materials may also be adapted for use with naturally occurring double stranded DNA fragments, such as restriction fragment length polynucleotides, which can be useful as, e.g., restriction fragment length polymorphisms (RFLPs). Such fragments may be included as all or part of a synthetic gene made according to the present process. That is, in addition to synthetic single stranded oligonucleotides, one may use a dsDNA fragments with overhangs, so-called “sticky ends,” as shown in FIG. 1B. Sticky end fragments 30, 32, as shown in FIG. 1B, can be generated with a number of restriction enzymes which create overhangs with defined lengths. As described below, the length of the overlaps I and I′ are calculated according to the methods described. One may find restriction enzymes that provide desired overhangs at, e.g., New England Biolabs “enzyme finder,” http://www.neb.com/nebecomm/EnzymeFinderSearchbyEnd.asp. These fragments may be joined with other synthetic oligos as described above. That is, one of the duplexes shown in FIG. 1B may be generated from naturally occurring dsDNA, and the other may be one of the duplexes created according to the steps described above.

These methods can be carried out using conventional (i.e., microliter) scale volumes or can be carried out at the nanoliter scale (or below) using microfluidic devices.

### Generalized Method and Apparatus

The strategy for designing oligonucleotides for assembly is implemented, in the preferred embodiment, by a computer program. The program used here was written in MATLAB, and the source code is given below, in APPENDIX 1. MATLAB is a numerical computing environment and programming language, which was created by The MathWorks. MATLAB allows easy matrix manipulation, plotting of functions and data, implementation of algorithms, creation of user interfaces, and interfacing with programs in other languages.

As explained above, the logic of this program is to find sequences of different lengths and melting temperatures (ligation temperatures) so that assembly is limited to specific fragments at defined temperature levels.

The MATLAB program is used to calculate the annealing temperature (melting temperature of DNA strands) of oligos, in order to calculate how long each oligo should be. Basically, one needs to design oligos that have two sections in each oligo. Each section must fit the requirement for the designed melting temperature so that at each stage the ligase will join short oligos together but any imperfectly matched ds-DNA will melt (not hybridize) and will not be able to stitch into a longer piece. The program only calculates the melting temp of each section. If the section is too short, then the program will add one nucleotide to make it longer then re-calculate the melting temp. If the section is too long, then the program will delete one nucleotide and re-calculate. This is repeated until the right length of the desired section is obtained. This procedure is then repeated to calculate the next section. This can be done by hand, but using the above program makes it much faster.

A variety of methods may be used to calculate DNA melting temperature. One common method, considered the fastest, is the Wallace method, using the formula Tₘ=2(A+T)+4(G+C).

For an aqueous solution of DNA (no salt) another formula for Tm is:

Tm=69.3° C.+0.41(% G+C)° C.

Under salt-containing hybridization conditions, the formula for the Effective Tm (Eff Tm) is as follows.

Eff Tm=81.5+16.6(log M[Na+])+0.41(% G+C)−0.72(% formamide).

Online tools are available for entering sequences and calculating their melting temperature. See for example the website at http://www.idtdna.com/analyzer/Applications/OligoAnalyzer/. For example, taking the underlined sequence below, one may calculate, for those 30 bases, a 50.0% GC content and a melting temperature of 63.0° C. Removing the last three bases changes the temperature to 60.9° C., since, as is known, shorter strands have less hybridization potential and are easier to separate.

In order to construct a gene of approximately 1,000 bp, as in the example below, one can design a protocol as follows:

First, one establishes an approximate length for constructing oligonucleotides on the order of 15 to 45 bases long. The length is determined by convenience and the cost of small oligonucleotides, which generally increases as lengths exceed 30 nt. In this case, a length of 30-40 nt was used, with about 15-20 base overlap on either end. This overlap will determine the melting temperature of the first round of ligation, with some variation (plus or minus 2 degrees) permitted.

In this case, a final length of about 1,000 nt is desired, so divided by 30 nt in length=about 27 pools of constructing oligonucleotide. Each pool will have 3 oligonucleotides for ligation as shown in FIG. 1. The ligated polynucleotides from one round are passed on to the next round, as shown in Table 1. That is, Round 2 is comprised of 9 pools of 3 polynucleotides selected from among the 27 pools of Round 1.

TABLE 1

| | | Number of pools of | Number of |
|---|---|---|---|
| | | polynucleotides to | polynucleotides |
| Round | Temperature, ° C. | be ligated | in each pool |
| 1 | 50 | 27 | 3 |
| 2 | 55 | 9 | 3 |
| 3 | 60 | 3 | 3 |
| 4 | 65 | 1 | 3 |

The above protocol simplifies the design of the constructing subsequences because the overlap regions do not have to be unique from pool to pool. However, it is possible to combine different polynucleotides in the same pool if unique overlap regions are designed, so that, as in FIG. 1, A will only hybridize to A′, B to B′, D to D′, E to E′, etc. As can be seen, the number of pools is chosen so that in round 1, the resultant duplexes are sufficient to, upon further rounds of hybridization/ligation, form the complete sequence.

### EXAMPLES

### Example 1

### Design of Constructing Fragments

The following sequence (SEQ ID NO: 1) (having 1008 nt) was constructed

atgaaacgtg taaaaccgct aagaaggtgg tgg|ccgggta caccttctac ccgctc|tgtc aaatcttctg tgactacgaa
ccttccgtaa tgcgctgaac tcgtatcgaa tgctgcgttg gaagaaggtga ggtatcatctc aaatgcctgtc acctctatcga
gaagacatcct tctcacttctt tgccgtgttgt tgctctcagga gcttggaacgc gtttggttcgt aacgctgaagt aacctgctgcg
ctgcgtatctc aaaaacccggg ggtgctcgtgc ttcttcctgtc gaagttctgcc cacatggacgg gctcgtaccca gacggtgttct
gttctgggtat cgttctcgtta aacccggacgt gttgcttctgc gcttcttctga gttctggttca ttcaccaaagt atcgacctgat
ggtaaacgtgc gttgttccg aaggtggtta ctgcgtctga cgacgaaggt tcggttctct ctggtttctg attccgtcgt acctgtctaa
cgtatggacg actggttgaa tctcttctaa gaaccggacg acgtatgcgt ttctgtctac ctctctgctg gctaacccga
ccggttacga acgtggtacc aacttctctc tggaatcttc tgaacgttac ttcccgccgt gcatgcagga catcatggaa
cgtctgcgta aaaacaaaca cctgaaatac aacgaccgtc agaccctgtg cctgttcctg aaagactgcg gtatgtctgt
tgaagacggt atcgctttct tccgtggttc tttcaaagct ccgcgtgaag ttttcgacaa agaatacctg tactctatcc gtcacaacta
cggtctggaa ggtaaacgtg ctaactactc ttgcttctct tgctctaaaa tcgctaacat gaccaacgaa gaacgtcgtt cttcttgccc
gttcgttggt gacccggaac acatccgtga acgtatcaaa gacgttggtg ctgacatcga agacatcatg ggtgaaggtc
cgttcaacgg taaatgcacc cgtctgctgg aaaaactgac ctgccgtaaa cagtctcgtc tggttgctac cccggttcgt
tactacctgg aatgcaaaga cccggaacgt aacggtggtg aaatctaa

For purposes of illustration, sequences S01 and S02 from appendix I are set off by bars.

This translates in frame 1 to: (SEQ ID NO: 2)

MKRAKTAKEGGWPGITFYTALSKSSVDYETFRKCAERRIEMLRCEEGDGI
ISKCLSTSIEEDILSHFFCRVVCSQDAWNAVWFVNAEVNLLRLRISKNPG
GARAFFLSEVLPHMDGARTQDGVLVLGMRSRYNPDVVASAASSEVLVHFT
KVIDLIGKRAVVPEGGYLRLNDEGIGSLLVSEFRRYLSNRMDELVEISSN
EPDERMRVLSTSLLANPTGYERGTNFSLESSERYFPPCMQDIMERLRKNK
HLKYNDRQTLCLFLKDCGMSVEDGIAFFRGSFKAPREVFDKEYLYSIRHN
YGLEGKRANYSCFSCSKIANMTNEERRSSCPFVGDPEHIRERIKDVGADI
EDIMGEGPFNGKCTRLLEKLTCRKQSRLVATPVRYYLECKDPERNGGEI*

This corresponds at the amino acid level to DNA polymerase alpha/primase large subunit, e.g., GenBank Locus NP_{—}597464. However, the codon usage in the above DNA sequence has been optimized for expression in E. coli, so the present DNA sequence does not exist in nature, and must be created artificially. The human version of this enzyme is further described at Stadlbauer, F., Brueckner, A., Rehfuess, C., Eckerskorn, C., Lottspeich, F., Forster, V., Tseng, B. Y. and Nasheuer, H. P., “DNA replication in vitro by recombinant DNA-polymerase-alpha-primase,” Eur. J. Biochem., 1994, 222 (3), 781-793.

### Example 2

### Gene Synthesis by 4-Stage Ligation

We used T4 polynucleotide kinase (PNK) from New England Biolabs to phosphorylate the constructing oligonucleotides. The reaction followed the protocol suggested by the manufacturer.

Stage 1 ligation. We divided the 80 constructing oligonucleotides into 27 pools (most pools contain 3 oligonucleotides, only one contains 2 oligonucleotides). For each pool, we picked up 15 uL 4 uM phosphorylated oligonucleotides into a 0.2 mL tube, then added 6 uL 10× Ampligase reaction buffer (EpiBio) and 3 uL water. We set the temperature to 50° C., then added 6 uL (30 U) Ampligase (EpiBio) into each tube and incubated for 1 hour. The complete list of oligos used is given in APPENDIX II, which shows the orientation of the oligo with regard to the final sequence, and the melting temperature for the hybridization with the next oligo in the synthesis.

Stage 2 ligation. On the thermocycler, we raised the temperature to 55° C., picked 20 uL of each reaction solution from stage 1, and combined 3 of them into a new tube according to the design. The total tube number was 9 in this stage. For each tube, 10 uL of ligase (50 U) was added. The tubes were incubated for 1 hour.

Stage 3 ligation. On the thermocycler, we raised the temperature to 60° C., picked 20 uL of each reaction solution from stage 2, and combined 3 of them into a new tube according to the design. The total tube number was 3 in this stage. For each tube, 10 uL of ligase (50 U) was added. The tubes were incubated for 1 hour.

Stage 4 ligation. On the thermocycler, we raised the temperature to 65° C., picked 20 uL of each reaction solution from stage 3, and combined them into a new tube. Then 10 uL of ligase (50 U) was added. The tube was incubated for 1 hour.

### Example 3

### Gene Synthesis by One-Stage Ligation. (Comparative Example)

We used 1 uL of each constructing oligonucleotide (4 uM) and added 10 uL ligase buffer. We heated the mix to 55° C. and then added 10 uL Ampligase (50 U) (EpiBio), incubated at 55° C. for 2 hours.

### Example 4

### PCR Amplification of Ligated Product

The short oligonucleotides in the ligation product were cleaned using a Montage spin-column. The long oligonucleotides left on the membrane were re-suspended in 20 uL water. PCR amplification was done under following condition:

1 uL cleaned ligation product as templates. 0.5 uL dNTP (10 mM each). 0.5 uL primer mix (50 uM for each primer). 5 uL PCR buffer (Roche expand high fidelity PCR system). 18 uL water. 0.25 uL polymerase mix (0.88 U, Taq polymerase with Tgo polymerase, Expand High Fidelity PCR System, Roche Applied Science).

45 cycles. Each cycle: 94° C. 30 sec, 58.5° C. 60 sec, 72° C. 90 sec.

### Example 5

### Demonstration of Purity of Ligation Product

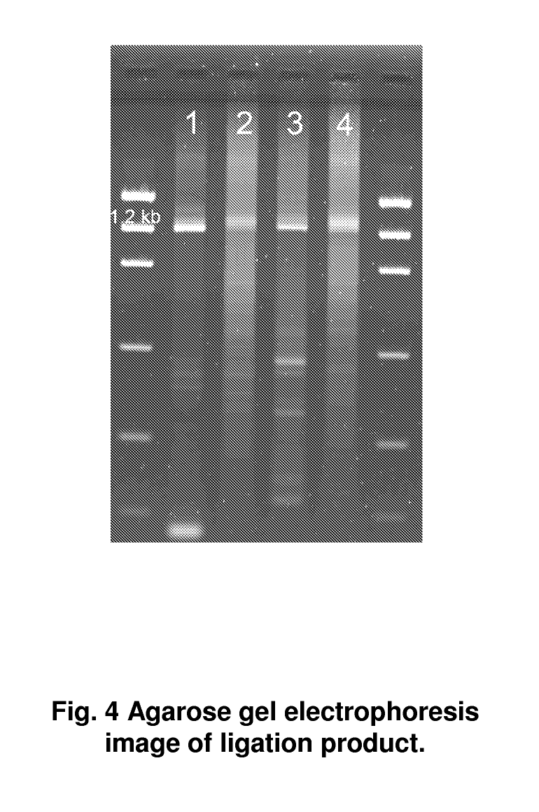

FIG. 4 shows the difference between the multi-stage ligation gene synthesis and the single-stage ligation method. Lanes 1 and 3 are a 1.2 kb gene made by 4-stage ligation (from 80 constructing oligonucleotides) and then PCR amplified using different primers. Lanes 2 and 4 are the same gene made by single-stage ligation and then PCR amplified using corresponding primers. Clearly, the 4-stage ligation strategy generates a much cleaner gene product than the single-stage method.

### CONCLUSION

The above specific description is meant to exemplify and illustrate the invention and should not be seen as limiting the scope of the invention, which is defined by the literal and equivalent scope of the appended claims. Any patents or publications mentioned in this specification are indicative of levels of those skilled in the art to which the patent pertains and are intended to convey details of the invention which may not be explicitly set out but which would be understood by workers in the field. Such patents or publications are hereby incorporated by reference to the same extent as if each was specifically and individually incorporated by reference, as needed for the purpose of describing and enabling the method or material referred to.

```
APPENDIX I
clear;
[header0, fullseq_sense] = fastaread(‘EcunPri2.fasta’);
full_length = length(fullseq_sense);
CP = int16(full_length/2); % define center point.
T1 = 45;T2 = T1+5;T3 = T2+5;T4 = T3+5;
T_err = 2.0;
X = ‘abaacaabaabaa’;x = rot90(X)’;cut = strcat(X, ‘d’, x, X, ‘d’);
%right side.....
indi = CP; ii = 0;
while indi < full_length
  ii = ii +1;
  indi;
  n = cut(ii);
  switch n
  case {‘a’}
  OL(ii) = 11; Td(ii) = T1;Co(ii)=1;
  case {‘b’}
  OL(ii) = 15; Td(ii) = T2;Co(ii)=2;
  case {‘c’}
  OL(ii) = 20; Td(ii) = T3;Co(ii)=3;
  case {‘d’}
  OL(ii) = 22; Td(ii) = T4;Co(ii)=4;
  otherwise
  OL(ii) = 0; Td(ii) = 0;
  end
  nextindi = indi+OL(ii);
  if nextindi > full_length+1
  nextindi = full_length+1;
  Oligo_r{ii} = fullseq_sense(indi:nextindi−1);
  else
  Oligo_r{ii} = fullseq_sense(indi:nextindi−1);
  A = oligoprop(Oligo_r{ii});
  T_m(ii) = A.Tm(5);
  while abs(T_m(ii)−Td(ii)) > T_err
  if T_m(ii)−Td(ii) > T_err
  nextindi = nextindi − 1;
  Oligo_r{ii} = fullseq_sense(indi:nextindi−1);
  A = oligoprop(Oligo_r{ii});
  T_m(ii) = A.Tm(5);
  else
  nextindi = nextindi + 1;
  Oligo_r{ii} = fullseq_sense(indi:nextindi−1);
  A = oligoprop(Oligo_r{ii});
  T_m(ii) = A.Tm(5);
  end
  end
  end
  indi = nextindi;
  OligoLength_r(ii) = length(Oligo_r{ii});
end
figure(1);
h=bar(T_m);ylim([42 62]);title(‘RIGHT strands’);xlim([0 length(T_m)]);
xlabel(‘strand # (center -> right)’);ylabel(‘melting temp. (dC)’);
line([0 40],[T1 T1]);
line([0 40],[T2 T2]);
line([0 40],[T3 T3]);
line([0 40],[T4 T4]);
ch = get(h,‘Children’);
fvd = get(ch,‘Faces’);
fvcd = get(ch,‘FaceVertexCData’);
for i = 1:length(T_m)
  fvcd(fvd(i,:)) = Co(i);
end
set(ch,‘FaceVertexCData’,fvcd)
%right side finished .......
% left side .....
indi = CP; ii = 0;
while indi > 1
  ii = ii +1;
  n = cut(ii);
  switch n
  case {‘a’}
  OL(ii) = 13; Td(ii) = T1; Co_l(ii)=1;
  case {‘b’}
  OL(ii) = 18; Td(ii) = T2; Co_l(ii)=2;
  case {‘c’}
  OL(ii) = 23; Td(ii) = T3; Co_l(ii)=3;
  case {‘d’}
  OL(ii) = 26; Td(ii) = T4; Co_1(ii)=4;
  otherwise
  OL(ii) = 0; Td(ii) = 0;
  end
  nextindi = indi−OL(ii);
  if nextindi < 0
  nextindi = 1;
  Oligo_l{ii} = fullseq_sense(nextindi:indi−1);
  else
  Oligo_l{ii} = fullseq_sense(nextindi:indi−1);
  A = oligoprop(Oligo_l{ii});
  T_m_l(ii) = A.Tm(5);
  while abs(T_m_l(ii)−Td(ii)) > T_err
  if T_m_l(ii)−Td(ii) > T_err
  nextindi = nextindi + 1;
  Oligo_l{ii} = fullseq_sense(nextindi:indi−1);
  A = oligoprop(Oligo_l{ii});
  T_m_l(ii) = A.Tm(5);
  else
  nextindi = nextindi − 1;
  Oligo_l{ii} = fullseq_sense(nextindi:indi−1);
  A = oligoprop(Oligo_l{ii});
  T_m_l(ii) = A.Tm(5);
  end
  end
  end
  indi = nextindi;
  Oligo_l(ii);
  OligoLength_l(ii) = length(Oligo_l{ii});
end
LL = length(Oligo_l); LLT = length(T_m_l);
if LL ~= LLT
  A = oligoprop(Oligo_l{LL});
  T_m_l(LL) = A.Tm(5);
end
figure(2);
h = bar(T_m_l);ylim([42 62]);title(‘LEFT Strands’);xlim([0 length(T_m_l)]);
xlabel(‘strand # (center -> left)’);ylabel(‘melting temp. (dC)’);
line([0 40],[T1 T1]);
line([0 40],[T2 T2]);
line([0 40],[T3 T3]);
line([0 40],[T4 T4]);
ch = get(h,‘Children’);
fvd = get(ch,‘Faces’);
  fvcd = get(ch,‘FaceVertexCData’);
for i = 1:length(T_m_l)
fvcd(fvd(i,:)) = Co(i);
end
set(ch,‘FaceVertexCData’,fvcd)
% for ii = 1:length(Oligo_l)
% A = oligoprop(Oligo_l{ii});
% f_gc_l(ii) = A.GC;
% end
%
% for ii = 1:length(Oligo_r)
% A = oligoprop(Oligo_r{ii});
% f_gc_r(ii) = A.GC;
% end
% combine the fragments to oligoes for the Sense-strand, 5′ -> 3′
iiL = 0;
for ii = 3:2:length(Oligo_l)
  iiL = iiL+1;
  OL = strcat(Oligo_l{ii},Oligo_l{ii−1});
  OOL(iiL)=cellstr(OL);
end
OOO = rot90(OOL);
if ii == length(Oligo_l)−1
  OOO{1} = strcat(Oligo_l{length(Oligo_l)},OOO{1});
end
OO_center = strcat(Oligo_l{1},Oligo_r{1});
OOO{iiL+1} =OO_center;
centerstrandpoint = iiL+1;
iiR = 0;
for ii = 3:2:length(Oligo_r)
  iiR = iiR+1;
  OR = strcat(Oligo_r{ii−1},Oligo_r{ii});
  OOR(iiR)=cellstr(OR);
end
if ii < length(Oligo_r)
  OOR(iiR+1)=cellstr(Oligo_r(length(Oligo_r)));
end
Otemp = OOR′;
for ii = 1:length(Otemp)
  OOO{iiL+1+ii} = Otemp{ii};
end
OOO;
len = 0;
for ii = 1:length(OOO)
  len = len + length(OOO{ii});
end
if len ~= full_length
  error(‘something wrong’);
end
% figure out the Oligoes for generate the complementary strand
iiL = 0;
for ii = 1:2:length(Oligo_l)−1
  iiL = iiL+1;
  OL = strcat(Oligo_l{ii+1},Oligo_l{ii});
  OOL(iiL)=cellstr(OL);
end
if ii ~= length(Oligo_l)−1
  iiL = iiL + 1;
  OOL{iiL} = Oligo_l{length(Oligo_l)};
end
OOT = rot90(OOL);
consensebreakpoint = iiL;
iiR = 0;
for ii = 1:2:length(Oligo_r)−1
  iiR = iiR+1;
  OR = strcat(Oligo_r{ii},Oligo_r{ii+1});
  OOR(iiR)=cellstr(OR);
end
if ii ~= length(Oligo_r)−1
  OOR(iiR+1)=cellstr(Oligo_r(length(Oligo_r)));
end
Otemp = OOR′;
for ii = 1:length(Otemp)
  OOT{iiL+ii} = Otemp{ii};
end
for ii = 1:length(OOT)
  OC = seqrcomplement(OOT{ii});
  OC1 = seqcomplement(OOT{ii});
  OOCT(ii) = cellstr(OC);
  OOCT1(ii) = cellstr(OC1);
end
OOC=OOCT′;
OOC1 = OOCT1′;
length(OOO);
length(OOC);
len = 0;
for ii = 1:length(OOC)
  len = len + length(OOC{ii});
end
if len ~= full_length
  error(‘something wrong’);
end
%build a table containing the fragments (Oligo_l and Oligo_r),
%the melting temp for each fragments, the code for temp
%final_table(:,1) = Oligo_l;
%final_table(:,2) = cellstr(T_m_l);
%final table(:,3) = Co;
% generate pool strands in sense stand 5′ -> 3′
% ----left side:
ii = centerstrandpoint;
jj = 0;
pool = 1;
while ii > 0
  if pool == 1
  jj = jj +1;
  PO{jj} = OOO{ii};
  PO_disp{jj} = PO{jj};
  sn(jj) = 1;
  lbl{jj} = strcat(‘sense #’,num2str(ii));
  ii = ii −1;
  pool = 2;
  else if ii == 1
  jj = jj +1;
  PO{jj} = OOO{1};
  PO_disp{jj} = PO{jj};
  lbl{jj} = strcat(‘sense #’,num2str(ii));
  sn(jj) = 1;
  ii = ii −2;
  else
  jj = jj +1;
  PO{jj} = strcat(OOO{ii−1},OOO{ii});
  PO_disp{jj} = strcat(OOO{ii−1},‘|’,OOO{ii});
  lbl{jj} = strcat(‘sense #’,num2str(ii−1), ‘ AND sense #’,num2str(ii));
  sn(jj) = 2;
  ii = ii −2;
  pool = 1;
  end
  end
end
PO = rot90(PO);
PO_disp = rot90(PO_disp);
sn = rot90(sn);
lbl= rot90(lbl);
% ----right side
ii = centerstrandpoint+1;
jj = length(PO); center_pool_indi = jj;
pool = 2;
while ii <= length(OOO)
  if pool == 1
  jj = jj +1;
  PO{jj} = OOO{ii};
  PO_disp{jj} = PO{jj};
  sn(jj) = 1;
  lbl{jj} = strcat(‘sense #’,num2str(ii));
  ii = ii +1;
  pool = 2;
  else if ii == length(OOO)
  jj = jj + 1;
  PO{jj} = OOO{ii};
  PO_disp{jj} = PO{jj}
  sn(jj) = 1;
  lbl{jj} = strcat(‘sense #’,num2str(ii));
  ii = ii +2;
  else
  jj = jj +1;
  PO{jj} = strcat(OOO{ii},OOO{ii+1});
  PO_disp{jj} = strcat(OOO{ii},‘|’,OOO{ii+1});
  sn(jj) = 2;
  lbl{jj} = strcat(‘sense #’,num2str(ii), ‘ AND sense #’,num2str(ii+1));
  ii = ii +2;
  pool = 1;
  end
  end
end
PO_disp;
lbl;
% % generate pool strands in consense stand 3′ → 5′
% ----left side:
% OOT: calculate the oligoes using sense stand
% OOC1: the complementary strands 3′ → 5′
% OOC: the complementary strands 5′ → 3′
ii = consensebreakpoint−1;
jj = 0;
pool = 1;
while ii > 0
  if pool == 1
  jj = jj +1;
  POC{jj} = OOC1{ii};
  POC_disp{jj} = POC{jj};
  snc(jj) = 1;
  lbl_c{jj} = strcat(‘antisense #’,num2str(ii));
  ii = ii −1;
  pool = 2;
  else if ii == 1
  jj = jj +1;
  POC{jj} = OOC1{1};
  POC_disp{jj} = POC{jj};
  lbl_c{jj} = strcat(‘antisense #’,num2str(ii));
  snc(jj) = 1;
  ii = ii −2;
  else
  jj = jj +1;
  POC{jj} = strcat(OOC1{ii−1},OOC1{ii});
  POC_disp{jj} = strcat(OOC1{ii−1},‘|’,OOC1{ii});
  lbl_c{jj} = strcat(‘antisense #’,num2str(ii−1), ‘ AND antisense
#’,num2str(ii));
  snc(jj) = 2;
  ii = ii −2;
  pool = 1;
  end
  end
end
POC = rot90(POC);
POC_disp = rot90(POC_disp);
snc = rot90(snc);
lbl_c= rot90(lbl_c);
%---- center pool
jj = jj +1;
ii = consensebreakpoint;
POC{jj} = strcat(OOC1{ii},OOC1{ii+1});
POC_disp{jj} = strcat(OOC1{ii},‘|’,OOC1{ii+1});
snc(jj) = 2;
lbl_c{jj} = strcat(‘antisense #’,num2str(ii), ‘ AND antisense
#’,num2str(ii+1));
if length(POC) ~= center_pool_indi
  error(‘not right! the pool numbers are not matched!!!’);
end
% ----right side
ii = centerstrandpoint+2;
jj = length(POC);
pool = 1;
while ii <= length(OOC1)
  if pool == 1
  jj = jj +1;
  POC{jj} = OOC1{ii};
  POC_disp{jj} = POC{jj};
  snc(jj) = 1;
  lbl_c{jj} = strcat(‘antisense #’,num2str(ii));
  ii = ii +1;
  pool = 2;
  else if ii == length(OOC1)
  jj = jj + 1;
  POC{jj} = OOC1{ii};
  POC_disp{jj} = POC{jj};
  snc(jj) = 1;
  lbl_c{jj} = strcat(‘antisense #’,num2str(ii));
  ii = ii +2;
  else
  jj = jj +1;
  POC{jj} = strcat(OOC1{ii},OOC1{ii+1});
  POC_disp{jj} = strcat(OOC1{ii},‘|’,OOC1{ii+1});
  snc(jj) = 2;
  lbl_c{jj} = strcat(‘antisense #’,num2str(ii), ‘ AND antisense
#’,num2str(ii+1));
  ii = ii +2;
  pool = 1;
  end
  end
end
POC_disp;
lbl_c;
% finish generate pools
% display each pool
mm = min(length(PO),length(POC));
if length(PO) ~= mm
  disp(‘*******************************************************************’);
  disp(‘CAUTION: sense strands need more pools!’);
  disp(‘*******************************************************************’);
  casenum = 1;
else if length(POC) ~= mm
  disp(‘*******************************************************************’);
  disp(‘CAUTION: antisense strands need more pools’);
  disp(‘*******************************************************************’);
  casenum = 2;
  end
end
% for ii = 1:3
% K = strcat(‘Pool # ’, num2str(ii));
% disp(K);
% disp(lbl_c{ii});
% disp(POC_disp{ii});
% %disp(blanks(1)’);
% disp(PO_disp{ii});
% disp(lbl{ii});
% disp(blanks(5)’);
% end
for ii = 1:27
  U = POC{ii}; U1 = POC_disp{ii};
  D = PO{ii}; D1 = PO_disp{ii};
  L_U = length(U); L_D = length(D);
  sp1 = ‘ ’;
  sp2 = ‘ ’;
  if L_U > L_D % case: antisense is longer
  U_C = seqcomplement(U); % the complement of antisense seq
  p1 = findstr(U_C, D);
  p2 = findstr(U1, ‘|’);
  p3 = p1 + L_D;
  BH = blanks(p1−1);
  ins_p = p2 − p1;
  D2l = D(1:ins_p);
  D2r = D(ins_p+1:L_D);
  D_ml = seqcomplement(U(1:p1−1));
  D_mr = seqcomplement(U(p3:L_U));
  A1 = oligoprop(D_ml); t1 = A1.Tm(5);
  A2 = oligoprop(D2l); t2 = A2.Tm(5);
  A3 = oligoprop(D2r); t3 = A3.Tm(5);
  A4 = oligoprop(D_mr); t4 = A4.Tm(5);
  sprintf(‘%s%d\n %s \n\n %s%2.2f %s%2.2f %s%2.2f %s%2.2f \n%s%s%s
\n%s%s%s%s%s%s\n\n %s’,...
  ‘Pool #’, ii, lbl_c{ii}, sp1, t1, sp2, t2, sp2, t3, sp2, t4,...
  ‘3’‘-’, U1, ‘−5’‘’, BH, ‘5’‘-’, ...
  D2l, ‘-’, D2r, ‘−3’‘’, lbl{ii})
  else % means L_U < L_D, with longer sense sequences.
  D_C = seqcomplement(D); % the complement of sense seq
  p1 = findstr(D_C, U);
  p2 = findstr(D1, ‘|’);
  p3 = p1 + L_U;
  BH = blanks(p1−1);
  if p2
  ins_p = p2−p1;
  U2l = U(1:ins_p); D_l = seqcomplement(U2l);
  U2r = U(ins_p+1:L_U); D_r = seqcomplement(U2r);
  D_ll = D(1:p1−1);
  D_rr = D(p3:L_D);
  A1 = oligoprop(D_ll); t1 = A1.Tm(5);
  A2 = oligoprop(D_l); t2 = A2.Tm(5);
  A3 = oligoprop(D_r); t3 = A3.Tm(5);
  A4 = oligoprop(D_rr); t4 = A4.Tm(5);
  sprintf(‘%s%d\n %s \n\n %s%2.2f %s%2.2f %s%2.2f %s%2.2f \n%s%s%s%s%s%s
\n%s%s%s\n\n %s’,...
  ‘Pool #’, ii, lbl_c{ii}, sp1, t1, sp2, t2, sp2, t3, sp2, t4,...
  BH, ‘3’‘-’, U2l, ‘-’, U2r, ‘−5’‘’, ‘5’‘-’, D1, ‘−3’‘’, lbl{ii})
  else
  if p1 == 1
  A1 = oligoprop(D(1:p3−1));
  A2 = oligoprop(D(p3:L_D));
  t1 = A1.Tm(5);
  t2 = A2.Tm(5);
  sprintf(‘%s%d\n %s \n\n %s%2.2f %s%2.2f \n%s%s%s \n%s%s%s \n\n%s’, ...
  ‘Pool #’, ii, lbl_c{ii}, sp2, t1, sp2, t2,...
  ‘3’‘-’, U, ‘−5’‘’, ‘5’‘-’, D, ‘−3’‘’, lbl{ii})
  else
  end
  end
  end
end
% Double check the sequence of the strands.
% 1. the OOO vs. fullseq_sense
comb = [ ];
for ii = 1:length(OOO);
  comb = strcat(comb, OOO{ii});
end
if strcmp(fullseq_sense, comb)
  disp(‘the sequences of the sense fragments ‘‘OOO’’ OK’)
end
comb1 = [ ];
for ii = length(OOC):1
  comb1 = strcat(comb1, OOC{ii});
end
if strcat(seqrcomplement(fullseq_sense), comb1)
  disp(‘the sequences of the antisense fragments ‘‘OOC’’ OK’)
end
++++++++++++++++++++++++++++++++++++++++++++++++++++++++++++++++++++
++++++++++++++++++++++++++++++++++++++++++++++++++++++++++++++++++++
```

APPENDIX II

| | SEQ | | | |
|---|---|---|---|---|
| Oligo | ID NO: | Position | Sequence | Tm |
| S01 | 3 | A1 | ATG AAA CGT GCT AAA ACC GCT AAA GAA GGT GGT TGG | 64.23° C. |
| S02 | 4 | B1 | CCG GGT ATC ACC TTC TAC ACC GCT C | 64.45° C. |
| S03 | 5 | C1 | TGT CTA AAT CTT CTG TTG ACT ACG AAA CCT TCC GT | 61.54° C. |
| S04 | 6 | D1 | AAA TGC GCT GAA CGT CGT ATC GAA ATG CTG CGT | 66.02° C. |
| S05 | 7 | E1 | TGC GAA GAA GGT GAC GGT ATC ATC TCT AA | 60.45° C. |
| S06 | 8 | F1 | ATG CCT GTC TAC CTC TAT CGA AGA AGA CAT CC | 61.57° C. |
| S07 | 9 | G1 | TGT CTC ACT TCT TCT GCC GTG TTG TTT GC | 62.61° C. |
| S08 | 10 | H1 | TCT CAG GAC GCT TGG AAC GCT GTT T | 62.57° C. |
| S09 | 11 | A2 | GGT TCG TTA ACG CTG AAG TTA ACC TGC TGC GTC T | 65.43° C. |
| S10 | 12 | B2 | GCG TAT CTC TAA AAA CCC GGG TGG TGC TC | 63.91° C. |
| S11 | 13 | C2 | GTG CTT TCT TCC TGT CTG AAG TTC TGC C | 61.18° C. |
| S12 | 14 | D2 | GCA CAT GGA CGG TGC TCG TAC CCA G | 65.32° C. |
| S13 | 15 | E2 | GAC GGT GTT CTG GTT CTG GGT ATG CGT TCT CGT TAC AAC | 66.46° C. |
| S14 | 16 | F2 | CCG GAC GTT GTT GCT TCT GCT GCT | 64.03° C. |
| S15 | 17 | G2 | TCT TCT GAA GTT CTG GTT CAC TTC ACC AAA GTT | 61.02° C. |
| S16 | 18 | H2 | ATC GAC CTG ATC GGT AAA CGT GCT GTT GTT C | 63.14° C. |
| S17 | 19 | A3 | CGG AAG GTG GTT ACC TGC GTC TGA A | 62.58° C. |
| S18 | 20 | B3 | CGA CGA AGG TAT CGG TTC TCT GCT GGT TTC TGA ATT C | 64.48° C. |
| S19 | 21 | C3 | CGT CGT TAC CTG TCT AAC CGT ATG GAC GAA CT | 63.25° C. |
| S20 | 22 | D3 | GGT TGA AAT CTC TTC TAA CGA ACC GGA CG | 60.53° C. |
| S21 | 23 | E3 | AAC GTA TGC GTG TTC TGT CTA CCT CTC TGC | 62.70° C. |
| S22 | 24 | F3 | TGG CTA ACC CGA CCG GTT ACG AAC GTG G | 66.84° C. |
| S23 | 25 | G3 | TAC CAA CTT CTC TCT GGA ATC TTC TGA ACG | 59.29° C. |
| S24 | 26 | H3 | TTA CTT CCC GCC GTG CAT GCA GGA CA | 66.15° C. |
| S25 | 27 | A4 | TCA TGG AAC GTC TGC GTA AAA ACA AAC ACC | 61.17° C. |
| S26 | 28 | B4 | TGA AAT ACA ACG ACC GTC AGA CCC TGT | 61.40° C. |
| S27 | 29 | C4 | GCC TGT TCC TGA AAG ACT GCG GTA TGT CTG TTG AAG AC | 65.69° C. |
| S28 | 30 | D4 | GGT ATC GCT TTC TTC CGT GGT TCT TTC AAA GC | 62.41° C. |
| S29 | 31 | E4 | TCC GCG TGA AGT TTT CGA CAA AGA ATA CC | 60.76° C. |
| S30 | 32 | F4 | TGT ACT CTA TCC GTC ACA ACT ACG GTC TGG | 61.76° C. |
| S31 | 33 | G4 | AAG GTA AAC GTG CTA ACT ACT CTT GCT TCT CTT GC | 62.28° C. |
| S32 | 34 | H4 | TCT AAA ATC GCT AAC ATG ACC AAC GAA GAA C | 58.78° C. |
| S33 | 35 | A5 | GTC GTT CTT CTT GCC CGT TCG TTG GTG | 63.44° C. |
| S34 | 36 | B5 | ACC CGG AAC ACA TCC GTG AAC GTA TCA | 63.26° C. |
| S35 | 37 | C5 | AAG ACG TTG GTG CTG ACA TCG AAG ACA | 61.85° C. |
| S36 | 38 | D5 | TCA TGG GTG AAG GTC CGT TCA ACG GTA AAT GC | 64.68° C. |
| S37 | 39 | E5 | ACC CGT CTG CTG GAA AAA CTG ACC TGC | 64.60° C. |
| S38 | 40 | F5 | CGT AAA CAG TCT CGT CTG GTT GCT ACC | 60.59° C. |
| S39 | 41 | G5 | CCG GTT CGT TAC TAC CTG GAA TGC AAA GAC | 62.13° C. |
| S40 | 42 | H5 | CCG GAA CGT AAC GGT GGT GAA ATC TAA | 60.41° C. |
| A01 | 43 | A6 | GGT TTT AGC ACG TTT CAT | 48.01° C. |
| A02 | 44 | B6 | GGT GAT ACC CGG CCA ACC ACC TTC TTT AGC | 65.33° C. |
| A03 | 45 | C6 | TAG TCA ACA GAA GAT TTA GAC AGA GCG GTG TAG AA | 61.00° C. |
| A04 | 46 | D6 | GTT CAG CGC ATT TAC GGA AGG TTT CG | 60.46° C. |
| A05 | 47 | E6 | CAC CTT CTT CGC AAC GCA GCA TTT CGA TAC GAC | 64.95° C. |
| A06 | 48 | F6 | ATA GAG GTA GAC AGG CAT TTA GAG ATG ATA CCG T | 60.65° C. |
| A07 | 49 | G6 | AGA AGA AGT GAG ACA GGA TGT CTT CTT CG | 59.32° C. |
| A08 | 50 | H6 | AGC GTC CTG AGA GCA AAC AAC ACG GC | 65.07° C. |
| A09 | 51 | A7 | GTT AAC TTC AGC GTT AAC GAA CCA AAC AGC GTT CCA | 64.20° C. |
| A10 | 52 | B7 | GGT TTT TAG AGA TAC GCA GAC GCA GCA G | 61.06° C. |
| A11 | 53 | C7 | CAG GAA GAA AGC ACG AGC ACC ACC CG | 65.29° C. |
| A12 | 54 | D7 | ACC GTC CAT GTG CGG CAG AAC TTC AGA | 65.50° C. |
| A13 | 55 | E7 | CCA GAA CAC CGT CCT GGG TAC GAG C | 64.74° C. |
| A14 | 56 | F7 | AAC AAC GTC CGG GTT GTA ACG AGA ACG CAT ACC CAG AA | 67.53° C. |
| A15 | 57 | G7 | AAC CAG AAC TTC AGA AGA AGC AGC AGA AGC | 62.12° C. |
| A16 | 58 | H7 | CGA TCA GGT CGA TAA CTT TGG TGA AGT G | 59.02° C. |
| A17 | 59 | A8 | TAA CCA CCT TCC GGA ACA ACA GCA CGT TTA C | 63.37° C. |
| A18 | 60 | B8 | AGA GAA CCG ATA CCT TCG TCG TTC AGA CGC AGG | 65.95° C. |
| A19 | 61 | C8 | GAC AGG TAA CGA CGG AAT TCA GAA ACC AGC | 62.22° C. |
| A20 | 62 | D8 | TTA GAA GAG ATT TCA ACC AGT TCG TCC ATA CGG TTA | 61.27° C. |
| A21 | 63 | E8 | AGA ACA CGC ATA CGT TCG TCC GGT TCG | 64.06° C. |
| A22 | 64 | F8 | CGG GTT AGC CAG CAG AGA GGT AGA C | 62.25° C. |
| A23 | 65 | G8 | AGA GAG AAG TTG GTA CCA CGT TCG TAA CCG GT | 64.53° C. |
| A24 | 66 | H8 | ACG GCG GGA AGT AAC GTT CAG AAG ATT CC | 63.37° C. |
| A25 | 67 | A9 | AGA CGT TCC ATG ATG TCC TGC ATG C | 61.37° C. |
| A26 | 68 | B9 | GGT CGT TGT ATT TCA GGT GTT TGT TTT TAC GC | 60.13° C. |
| A27 | 69 | C9 | CCG CAG TCT TTC AGG AAC AGG CAC AGG GTC TGA C | 68.08° C. |
| A28 | 70 | D9 | GAA GAA AGC GAT ACC GTC TTC AAC AGA CAT A | 59.66° C. |
| A29 | 71 | E9 | CTT CAC GCG GAG CTT TGA AAG AAC CAC G | 63.35° C. |
| A30 | 72 | F9 | GTG ACG GAT AGA GTA CAG GTA TTC TTT GTC GAA AA | 60.38° C. |
| A31 | 73 | G9 | AGC ACG TTT ACC TTC CAG ACC GTA GTT | 61.41° C. |
| A32 | 74 | H9 | ATG TTA GCG ATT TTA GAG CAA GAG AAG CAA GAG TAG TT | 61.57° C. |
| A33 | 75 | A10 | GGC AAG AAG AAC GAC GTT CTT CGT TGG TC | 62.72° C. |
| A34 | 76 | B10 | GTG TTC CGG GTC ACC AAC GAA CG | 62.50° C. |
| A35 | 77 | C10 | GCA CCA ACG TCT TTG ATA CGT TCA CGG AT | 62.27° C. |
| A36 | 78 | D10 | CGG ACC TTC ACC CAT GAT GTC TTC GAT GTC A | 64.13° C. |
| A37 | 79 | E10 | AGC AGA CGG GTG CAT TTA CCG TTG AA | 62.63° C. |
| A38 | 80 | F10 | CGA GAC TGT TTA CGG CAG GTC AGT TTT TCC | 62.34° C. |
| A39 | 81 | G10 | GGT AGT AAC GAA CCG GGG TAG CAA CCA GA | 64.31° C. |
| A40 | 82 | H10 | TTA GAT TTC ACC ACC GTT ACG TTC CGG GTC TTT GCA TTC CA | 67.00° C. |

What is claimed is:

1. A method for assembling a double stranded polynucleic acid molecule of a defined sequence from a set S of single stranded oligonucleotides, comprising the steps of:
a) preparing a set S of oligonucleotides having 5′ end portions (hereafter 5′E) and 3′ end portions (hereafter 3′E)
b) wherein at least one of said 5′E and 3′E portions have a complementary sequence to another oligonucleotide in set S, said complementary sequence for each oligonucleotide comprised of a plurality of different sequences having different, discrete melting temperatures and thereby dividing set S into subsets S₁ through Sₙ each with a different, discrete melting point as between the end portions; and
c) sequentially combining and ligating oligonucleotides from subsets S₁ through Sₙ at a different temperature for each subset until said polynucleotide molecule is assembled into the full length double stranded polynucleic acid molecule from said oligonucleotides which are successively hybridized at said 5′E and 3′E portions at successive steps at different, elevated temperatures, and ligated at successive steps at an elevated temperature.

2. The method of claim 1 wherein said polynucleic acid molecule is DNA.

3. The method of claim 1 wherein said subsets of set S has pools containing three oligonucleotides which hybridize to produce a gap for ligation.

4. The method of claim 1 wherein said oligonucleotides in set S are, prior to any ligation, between about 20 and 100 nucleotides in length.

5. The method of claim 1 wherein said set S comprises at least 80 oligonucleotides prior to ligation.

6. The method of claim 1 wherein said ligating is accomplished using a thermostable ligase stable above 50° C.

7. The method of claim 6 wherein the step of sequentially combining and ligating takes place at preselected temperatures differing by at least 2° C. and in a range between 20° C. and 65° C.

8. The method of claim 7 wherein said temperatures differ by between 4 and 6° C.

9. The method of claim 1 wherein the sequences of end portions 5′E and 3′E of two oligonucleotides are immediately adjacent in the defined sequence of the double-stranded polynucleic acid to provide a point for ligation and said two oligonucleotides overhang a third, hybridized oligonucleotide.

10. The method of claim 9 wherein no individual nucleotides are added and no polymerase is added.

11. The method of claim 1 wherein non-natural nucleotides are contained in the oligonucleotides.

12. A method for synthesizing a polynucleotide from oligonucleotides, using a thermostable ligase, but without using a polymerase, comprising the steps of:
(a) hybridizing, at a first temperature, multiple pools single stranded oligonucleotides, wherein each pool contains three single stranded oligonucleotides to form a double stranded polynucleotide having adjacent oligonucleotides that can be ligated to form a duplex;
(b) ligating the adjacent oligonucleotides from step (a) to form a plurality of duplexes;
(c) hybridizing at a second temperature, different from the first temperature, multiple duplexes from step (b) wherein each duplex forms an elongated double stranded polynucleotide having adjacent oligonucleotides that can be ligated to form a duplex; and
(d) ligating the resulting adjacent oligonucleotides from step (c).

13. The method of claim 12 wherein said hybridizing is at a first temperature between 20° C. and 60° C., and the second temperature differs from the first temperature by about 2° C. to 6° C.

14. A set of oligonucleotides for assembling a double stranded polynucleic acid molecule of a defined sequence from a plurality of single stranded oligonucleotides, comprising a set S of oligonucleotides having 5′ end portions (5′E) and 3′ end portions (3′E) overlapping with one or two other oligonucleotides in Set S, the 5′E and 3′E portions having a sequence and length resulting in a plurality of different, discrete melting temperatures and thereby dividing set S into subsets S₁ through Sₙ.

15. The set of claim 14 comprising a number of oligonucleotides equal to about 1/30 of the number of nucleotides in the final polynucleic acid.

16. The set of claim 14 wherein the region of said overlapping is between 5 and 25 nucleotides.

17. A kit comprising the set of oligonucleotides of claim 14 and a thermostable ligase.

18. The kit of claim 14 further comprising a thermocycler.

19. A computer program for designing a plurality of oligonucleotides which, when assembled, form a user predefined polynucleic acid molecule of a predetermined sequence from the plurality of single stranded oligonucleotides, said program
a) determining a set S of oligonucleotides having length L and 5′ end portions (5′E) and 3′ end portions (3′E) overlapping with another oligonucleotide in set S, the 5′E and 3′E portions of each oligonucleotide having a sequence and length resulting in a plurality P of different, discrete melting temperatures and thereby dividing set S into subsets S₁ through Sₙ, each subset containing at least three oligonucleotides prior to ligation; so that
b) sequentially combining and ligating subsets S₁ through Sₙ at a different temperature for each subset results in assembly of said polynucleic acid molecule.
